(19) United States
(12) Patent Application Publication
(10) Pub. No.: US 2022/0387412 A1
(43) Pub. Date: Dec. 8, 2022

(54) Novel Superebastine Against Therapy Resistant Prostate Cancer

(71) Applicant: University of Kentucky Research Foundation, Lexington, KY (US)

(72) Inventors: Vivek M. Rangnekar, Nicholasville, KY (US); David S. Watt, Lexington, KY (US); Ravshan Burikhanov, Lexington, KY (US); Vitaliy Sviripa, Lexington, KY (US)

(21) Appl. No.: 17/770,267

(22) PCT Filed: Nov. 4, 2020
(86) PCT No.: PCT/US20/58922

§ 371 Date: Apr. 19, 2022

Related U.S. Application Data
(60) Provisional application No. 62/930,328, filed on Nov. 4, 2019.

(51) Int. Cl. A61K 31/45; A61P 35/00

(52) U.S. Cl. CPC A61K 31/45; A61P 35/00

(57) ABSTRACT

A composition and method for treating tumor cells are provided. The composition includes at least one ebastine derivative having a structure according to Formula I. The method include administering, to a patient in need thereof, an effective amount of the composition including at least one ebastine derivative having the structure according to Formula I.

### RELATED APPLICATIONS

This application claims the benefit of U.S. Provisional Application Ser. No. 62/930,328, filed Nov. 4, 2019, the entire disclosure of which is incorporated herein by this reference.

### GOVERNMENT INTEREST

This invention was made with government support under grant numbers RO1 CA187273, T32 CA165990, P30 CA177558, R21 CA179283, RO1 CA165469, P30 GM110787, R01 CA187273, and P01 NS097197 awarded by the National Institutes of Health (NIH), and grant number W81XWH-16-1-0635 awarded by the Department of Defense (DOD). The government has certain rights in the invention.

### TECHNICAL FIELD

The present disclosure is directed to compounds and methods for treating therapy resistant cancer cells. In particular, the disclosure is directed to compounds and methods for treating Abiraterone (ABT)- and Enzalutamide (ENZ)-resistant prostate cancer cells.

### BACKGROUND

Prostate cancer is one of the leading causes of cancer-related deaths in men in the United States. Androgen deprivation therapy (ADT) is the mainstay of prostate cancer treatment. However, about 30% of the patients show relapse of the disease within 3 years of this treatment, developing resistance to ADT and progressing to castration-resistant prostate cancer (CRPC).

CRPC develops due to the reactivation of the androgen receptor (AR) signaling pathway. As such, CRPC patients are typically treated with Abiraterone (ABT) and Enzalutamide (ENZ). ABT inhibits CYP17A which is critical for androgen synthesis from cholesterol in prostate cancer cells, and ENZ binds to the ligand binding pocket of the AR in prostate cancer cells and prevents the activation of AR-dependent cell survival and proliferation signaling pathways. Although ABT and ENZ increase the lifespan of patients with castration-resistant prostate cancer, these patients quickly develop resistance to this treatment as well, with the average increase in lifespan being 4-6 months.

Resistance to ABT is attributed to upregulated expression of full-length AR or ligand-independent AR splice variants, which lack the ligand binding domain and are constitutively active, as well as induction of steroidogenic genes (including CYP17A). ENZ resistance is attributed to AR splice variants, activation of the glucocorticoid receptor pathway, de novo synthesis of androgens from cholesterol, or mutations in AR that allow non-specific ligand binding to activate the AR signaling pathway. Besides taxanes, which may suffer from cross-resistance and, even if effective, have severe side effects, there are no effective options against ENZ-resistant prostate cancer especially in older men.

Accordingly, there remains a need for compounds and methods to overcome treatment resistance in prostate cancer.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a composition for treating tumor cells, the composition including at least one compound having the structure according to Formula 1:

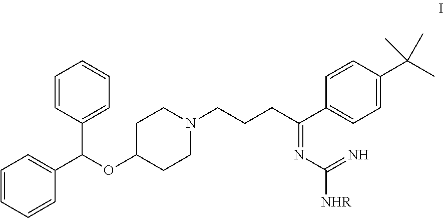

or an analog thereof, where R includes H, an alkyl group, a cycloalkyl group, an arylalkyl group, a heteroarylalkyl group, an acyl group, or a combination thereof. In some embodiments, the compound includes the structure:

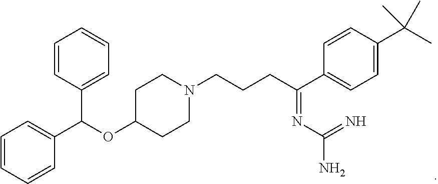 [SMILES: CC(C)(C)c1ccc(/C(CCCN2CCC(OC(c3ccccc3)c3ccccc3)CC2)=N\C(=N)N)cc1]

In some embodiments, R is an alkyl group. In some embodiments, R is a cycloalkyl group. In some embodiments, R is a benzylic group. In some embodiments, R is an arylalkyl group. In some embodiments, R is a heteroarylalkyl group. In some embodiments, R is an acyl group. In some embodiments, the composition includes at least two compounds having the structure according to Formula I. In some embodiments, the tumor cells are prostate tumor cells.

Also provided herein, in some embodiments, is a method of treating tumor cells, the method including administering, to a patient in need thereof, an effective amount of a composition including at least one compound having the structure according to Formula 1:

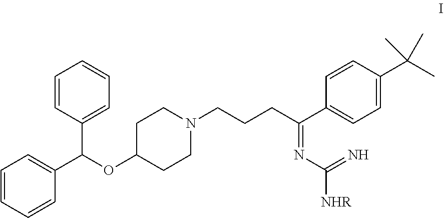

or an analog thereof, where R includes H, an alkyl group, a cycloalkyl group, an arylalkyl group, a heteroarylalkyl group, an acyl group, or a combination thereof. In some embodiments, the compound includes the structure:

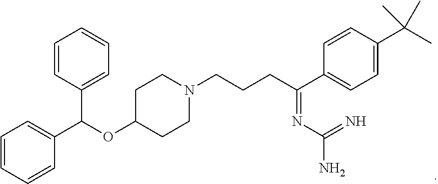 [SMILES: CC(C)(C)c1ccc(/C(CCCN2CCC(OC(c3ccccc3)c3ccccc3)CC2)=N\C(=N)N)cc1]

In some embodiments, R is an alkyl group. In some embodiments, R is a cycloalkyl group. In some embodiments, R is a benzylic group. In some embodiments, R is an arylalkyl group. In some embodiments, R is a heteroarylalkyl group. In some embodiments, R is an acyl group. In some embodiments, the composition includes at least two compounds having the structure according to Formula I. In some embodiments, the tumor cells are prostate tumor cells.

Further features and advantages of the presently-disclosed subject matter will become evident to those of ordinary skill in the art after a study of the description, figures, and non-limiting examples in this document.

### BRIEF DESCRIPTION OF THE DRAWINGS

FIGS. 1A-B show chemical structures of (A) ebastine (“EBS”) and (B) superebastine (“Super-EBS”).

FIGS. 2A-I show graphs illustrating that Super-EBS is more potent than EBS in inhibiting the viability of prostate cancer cells. (A-C) PC-3 cells treated with vehicle (V), EBS, or Super-EBS for (A) 24 h, (B) 48 h, or (C) 72 h, and then subjected to resazurin assays to test cell viability. (D-F) CWR22Rv1-ENZ-S cells treated with vehicle (V), EBS, or Super-EBS for (D) 24 h, (E) 48 h, or (F) 72 h, and then subjected to resazurin assays to test cell viability. (G-I) CWR22Rv1-ENZ-R cells treated with vehicle (V), EBS, or Super-EBS for (G) 24 h, (H) 48 h, and (I) 72 h, and then subjected to resazurin assays to test cell viability. Mean+SD shown. (*) Asterisk indicates P<0.0 by the Student's t-test.

FIG. 3 shows images and graphs illustrating that Super-EBS is more potent than EBS in inducing Caspase-3 and apoptosis of prostate cancer cells. Prostate cancer cells were treated with vehicle (V) or Super-EBS at the indicated concentrations for 24h, and then subjected to ICC for active caspase 3, and nuclei were stained with DAPI. Mean+SD shown. (*) Asterisk indicates P<0.0 by the Student's t-test.

FIGS. 4A-B show graphs illustrating viability of androgen independent and androgen dependent cells treated with Super-EBS. (A) Androgen independent AR-positive C4-2 and C4-2R were treated with Super-EBS and vehicle control for 24 h in 10% FBS. Cell viability was measured by Resazurin assay. (B) Androgen dependent AR-positive LNCaP and M49C were treated under same condition and cell viability by Resazurin assay. *P<0.05; ** P<0.01 by the Student's t test.

FIGS. 5A-B show images and graphs illustrating that Super-EBS causes cell death by the activation of an apoptotic cascade. (A) AR-v7 expressing CwR22Rv1 and CwR22Rv1 Enz(R) cells were treated with Super-EBS (0.1-5 μM) in 10% FBS for 24 h and tested by ICC analysis for cleaved caspase 3 (green) and DAPI (blue). (B) Quantitative representation of ICC data. *P<0.05; **P<0.01 by Student's t test.

FIG. 6 shows graphs illustrating a dose-dependent increase in the amount of cells in the early and late stages of apoptosis post-treatment with Super EBS. CwR22Rv1 Enz(R) cells were treated with Super-EBS (2 or 4 μM) for 24 h in 10% FBS containing medium. Live cells were subjected to Annexin V-PI staining and FACS analysis for apoptotic cells. The percentage on the figure indicates the percentage of cells post treatment.

FIGS. 7A-B show images illustrating that Super-EBS induces DNA damage in AR-positive prostate cancer cells. (A) C4-2 cells were treated with Super-EBS (0.5 or 1 μM) for 24 h in 10% FBS containing medium. (B) LNCaP cells were treated with Super-EBS (0.25 or 0.5 μM) and EBS (0.25-0.5 μM) for 24 h in 10% FBS containing medium. Lysates were probed for YH2AX (Ser139 phosphorylation), a marker for DSB by Western blot analysis.

FIGS. 8A-B show images illustrating that DNA damage induced by Super-EBS is mediated by Par-4. (A) CwR22Rv1 Par-4 wild type and knock-down cells were treated with Super-EBS (0.25 μM) for 24 h in medium containing 10% FBS, and then subjected to ICC for Par-4 (green) or YH2AX (Ser139) (red). (B) Western blot analysis for YH2AX, Par-4 and GAPDH was performed on lysates of Par-4 wild type (left) and knock-down (right) prostate cancer cells treated with Super-EBS (0.25 μM) for 24 h.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### Definitions

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below.

Following long-standing patent law convention, the terms “a,” “an,” and “the” refer to “one or more” when used in this application, including the claims. Thus, for example, reference to “a cell” includes a plurality of cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

All combinations of method or process steps as used herein can be performed in any order, unless otherwise specified or clearly implied to the contrary by the context in which the referenced combination is made.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, ElZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

As used herein, the term “patient” refers to a subject afflicted with a disease or disorder. A patient includes human and veterinary subjects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. It is also contemplated that, in certain aspects, unless expressly indicated to the contrary, individual substituents can be further optionally substituted (i.e., further substituted or unsubstituted).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The term “alkyl” as used herein is a branched or unbranched saturated hydrocarbon group of 1 to 24 carbon atoms, such as methyl, ethyl, propyl, isopropyl, butyl, isobutyl, s-butyl, i-butyl, pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group is acyclic. The alkyl group can be branched or unbranched. The alkyl group can also be substituted or unsubstituted. For example, the alkyl group can be substituted with one or more groups including, but not limited to, halogen, alkyl, cycloalkyl, aryl, heteroaryl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “arylalkyl” specifically refers to an alkyl group that is substituted with one or more aryl groups, as described below. The term “heteroarylalkyl” specifically refers to an alkyl group that is substituted with one or more heteroaryl groups, as described below, and the like. When “alkyl” is used in one instance and a specific term such as “haloalkyl” is used in another, it is not meant to imply that the term “alkyl” does not also refer to specific terms such as “haloalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, halogen, alkyl, cycloalkyl, aryl, heteroaryl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “aromatic group” as used herein refers to a ring structure having cyclic clouds of delocalized π electrons above and below the plane of the molecule, where the R clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The terms “halo,” “halogen,” or “halide,” as used herein can be used interchangeably and refer to F, Cl, Br, or I.

The term “heteroalkyl,” as used herein refers to an alkyl group containing at least one heteroatom. Suitable heteroatoms include, but are not limited to, O, N, Si, P and S, the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, pyrazolopyrimidinyl, 1,2-oxazol-4-yl, 1,2-oxazol-5-yl, 1,3-oxazolyl, 1,2,4-oxadiazol-5-yl, 1,2,3-triazolyl, 1,3-thiazol-4-yl, pyridinyl, and pyrimidin-5-yl.

The term “hydroxyl” as used herein is represented by the formula OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula N₃.

The term “nitro” as used herein is represented by the formula NO₂.

The term “nitrile” as used herein is represented by the formula CN.

The term “silyl” as used herein is represented by the formula SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas S(O)A¹, S(O)₂A¹, OS(O)₂A¹, or OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula SH.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

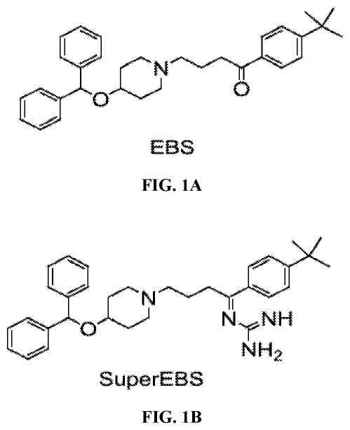

Provided herein are compounds for treating tumor cells, such as, but not limited to, prostate tumor cells, lung cancer cells, breast cancer cells, or a combination thereof. In some embodiments, the compounds include ebastine (4-[4-(Diphenylmethoxy)-1-piperidinyl]-1-[4-(2-methyl-2-propanyl)phenyl]-1-butanone; “EBS”) (FIG. 1A) and/or analogs thereof. In some embodiments, the compound includes a structure according to Formula 1:

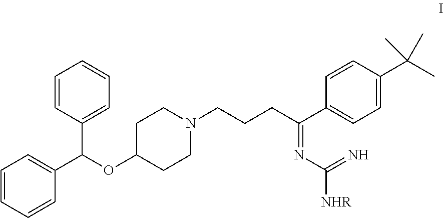

or an analog thereof, where R includes H, an alkyl group, a cycloalkyl group, an arylalkyl group, a heteroarylalkyl group, an acyl group, or a combination thereof.

In some embodiments, R includes H. In one embodiment, for example, the compound includes a guanidinylidene substituted EBS having the structure shown in FIG. 1B, which is referred to herein as superebastine or “Super-EBS.” Although discussed herein primarily with respect to Super-EBS, the disclosure is not so limited and expressly includes any other suitable EBS analog. In some embodiments, R includes an alkyl group. In some embodiments, R includes a cycloalkyl group. In some embodiments, R includes an arylalkyl group. In some embodiments, R includes a heteroalkyl group. In some embodiments, R includes an acyl group.

In some embodiments, R includes H, an alkyl group, or a combination thereof. In some embodiments, R includes H, a cycloalkyl group, or a combination thereof. In some embodiments, R includes H, an arylalkyl group, or a combination thereof. In some embodiments, R includes H, a heteroalkyl group, or a combination thereof. In some embodiments, R includes H, an acyl group, or a combination thereof. In some embodiments, R includes an alkyl group, a cycloalkyl group, or a combination thereof. In some embodiments, R includes an alkyl group, a an arylalkyl group, or a combination thereof. In some embodiments, R includes an alkyl group, a heteroalkyl group, or a combination thereof. In some embodiments, R includes an alkyl group, an acyl group, or a combination thereof. In some embodiments, R includes a cycloalkyl group, an arylalkyl group, or a combination thereof. In some embodiments, R includes a cycloalkyl group, a heteroalkyl group, or a combination thereof. In some embodiments, R includes a cycloalkyl group, an acyl group, or a combination thereof. In some embodiments, R includes an arylalkyl group, a heteroalkyl group, or a combination thereof. In some embodiments, R includes an arylalkyl group, an acyl group, or a combination thereof. In some embodiments, R includes a heteroalkyl group, an acyl group, or a combination thereof.

In some embodiments, R includes H, an alkyl group, a cycloalkyl group, or a combination thereof. In some embodiments, R includes H, an alkyl group, an arylalkyl group, or a combination thereof. In some embodiments, R includes H, a cycloalkyl group, an arylalkyl group, or a combination thereof. In some embodiments, R includes an alkyl group, a cycloalkyl group, an arylalkyl group, or a combination thereof. In some embodiments, R includes H, an alkyl group, a heteroalkyl group, or a combination thereof. In some embodiments, R includes H, a cycloalkyl group, a heteroalkyl group, or a combination thereof. In some embodiments, R includes an alkyl group, a cycloalkyl group, a heteroalkyl group, or a combination thereof. In some embodiments, R includes H, an arylalkyl group, a heteroalkyl group, or a combination thereof. In some embodiments, R includes an alkyl group, an arylalkyl group, a heteroalkyl group, or a combination thereof. In some embodiments, R includes a cycloalkyl group, an arylalkyl group, a heteroalkyl group, or a combination thereof. In some embodiments, R includes H, an alkyl group, an acyl group, or a combination thereof. In some embodiments, R includes H, a cycloalkyl group, an acyl group, or a combination thereof. In some embodiments, R includes an alkyl group, a cycloalkyl group, an acyl group, or a combination thereof. In some embodiments, R includes H, a heteroalkyl group, an acyl group, or a combination thereof. In some embodiments, R includes an alkyl group, a heteroalkyl group, an acyl group, or a combination thereof. In some embodiments, R includes an arylalkyl group, a heteroalkyl group, an acyl group, or a combination thereof.

In some embodiments, R includes H, an alkyl group, a cycloalkyl group, an arylalkyl group, or a combination thereof. In some embodiments, R includes H, an alkyl group, a cycloalkyl group, a heteroalkyl group, or a combination thereof. In some embodiments, R includes H, an alkyl group, an arylalkyl group, a heteroalkyl group, or a combination thereof. In some embodiments, R includes H, a cycloalkyl group, an arylalkyl group, a heteroalkyl group, or a combination thereof. In some embodiments, R includes an alkyl group, a cycloalkyl group, an arylalkyl group, a heteroalkyl group, or a combination thereof. In some embodiments, R includes H, an alkyl group, a cycloalkyl group, an acyl group, or a combination thereof. In some embodiments, R includes H, an alkyl group, an arylalkyl group, an acyl group, or a combination thereof. In some embodiments, R includes H, a cycloalkyl group, an arylalkyl group, an acyl group, or a combination thereof. In some embodiments, R includes an alkyl group, a cycloalkyl group, an arylalkyl group, an acyl group, or a combination thereof. In some embodiments, R includes H, an alkyl group, a heteroarylalkyl group, an acyl group, or a combination thereof. In some embodiments, R includes H, a cycloalkyl group, a heteroarylalkyl group, an acyl group, or a combination thereof. In some embodiments, R includes an alkyl group, a cycloalkyl group, a heteroarylalkyl group, an acyl group, or a combination thereof. In some embodiments, R includes H, an arylalkyl group, a heteroarylalkyl group, an acyl group, or a combination thereof. In some embodiments, R includes an alkyl group, an arylalkyl group, a heteroarylalkyl group, an acyl group, or a combination thereof. R includes a cycloalkyl group, an arylalkyl group, a heteroarylalkyl group, an acyl group, or a combination thereof.

In some embodiments, R includes H, an alkyl group, a cycloalkyl group, an arylalkyl group, a heteroalkyl group, or a combination thereof. In some embodiments, R includes H, an alkyl group, a cycloalkyl group, an arylalkyl group, an acyl group, or a combination thereof. In some embodiments, R includes H, an alkyl group, a cycloalkyl group, a heteroarylalkyl group, an acyl group, or a combination thereof. In some embodiments, R includes H, an alkyl group, an arylalkyl group, a heteroarylalkyl group, an acyl group, or a combination thereof. In some embodiments, R includes H, a cycloalkyl group, an arylalkyl group, a heteroarylalkyl group, an acyl group, or a combination thereof. In some embodiments, R includes an alkyl group, a cycloalkyl group, an arylalkyl group, a heteroarylalkyl group, an acyl group, or a combination thereof.

Also provided herein are methods for treating tumor cells, such as, but not limited to, prostate tumor cells. In some embodiments, the method includes administering an effective amount of one or more of the compounds disclosed herein to a patient in need thereof. For example, in one embodiment, the method includes administering an effective amount of EBS and/or one or more EBS analogs to a patient in need thereof. In another embodiment, the method includes administering an effective amount of Super-EBS and/or one or more other EBS analogs to a patient in need thereof. In a further embodiment, the method includes administering an effective amount of Super-EBS to a patient in need thereof. As will be understood from the “one or more” language used herein, the method expressly includes administering a single type of compound (e.g., Super-EBS or a single type of other EBS analog) or a combination of compounds disclosed herein (e.g., Super-EBS and EBS or any EBS analog).

The compounds and methods disclosed herein are suitable for treating previously untreated tumor cells as well as tumor cells that have become resistant to existing treatments. For example, in one embodiment, the compounds disclosed herein circumvent and/or overcome the existing drug resistance mechanisms in prostate tumor cells. In another embodiment, the compounds disclosed herein provide effective treatment against prostate cancer cells that are resistant to abiraterone (ABT) and/or enzalutamide (ENZ). In a further embodiment, the compounds disclosed herein induce apoptosis in ABT- and ENZ-resistant prostate cancer cells. Additionally or alternatively, in some embodiments, the compounds disclosed herein provide effective treatment for tumors, such as prostate tumors, that have metastasized to various tissues. Accordingly, in some embodiments, the methods disclosed herein include administering an effective amount of EBS and/or one or more EBS analogs to a patient that is resistant to ABT and/or ENZ treatment.

Administration can be by any method known to one of ordinary skill in the art. In some embodiments, suitable methods for administration of compounds of the present invention include, but are not limited to intravenous administration, bolus injection, and oral administration. Additionally or alternatively, the methods above may include administering a pharmaceutical composition comprising one or more of the compounds disclosed herein and a pharmaceutically acceptable carrier.

As used herein, the phrase “effective amount” refers to an amount of the present compounds and/or compositions that, when administered to a subject as a single dose or in multiple doses, leads to an amelioration of (e.g., an improvement of, a decreased duration of, etc.) at least one symptom of a disorder disclosed herein. In one embodiment, the disorder and/or the symptom is associated with tumor cells. In another embodiment, the disorder and/or the symptom is associated with prostate tumor cells. In a further embodiment, the effective amount reduces or eliminates proliferation of the tumor cells, reduces or eliminates metastasis of the tumor cells, induces apoptosis of the tumor cells, induces apoptosis of metastasized tumor cells, or combinations thereof in the subject.

The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed, and/or like factors well known in the medical arts. After review of the disclosure of the presently disclosed subject matter presented herein, one of ordinary skill in the art can tailor the dosages to an individual subject, taking into account the aforementioned factors. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

### Example 1

This Example describes the development and verification of novel compounds and methods for treating tumor cells. As there are currently no treatments available for ABT- or ENZ-resistant prostate cancer, the present inventors sought to identify small molecules that inhibit prostate cancer cells. The anti-histamine Ebastine (EBS), which is a second-generation H₁ receptor antagonist used mainly for allergic rhinitis and chronic idiopathic urticaria, was initially identified as a possible small molecule inhibitor of prostate cancer cells. EBS causes lysosomal catastrophe at high concentrations (LC₅₀ 18-22 μM) in NSCLC cells.

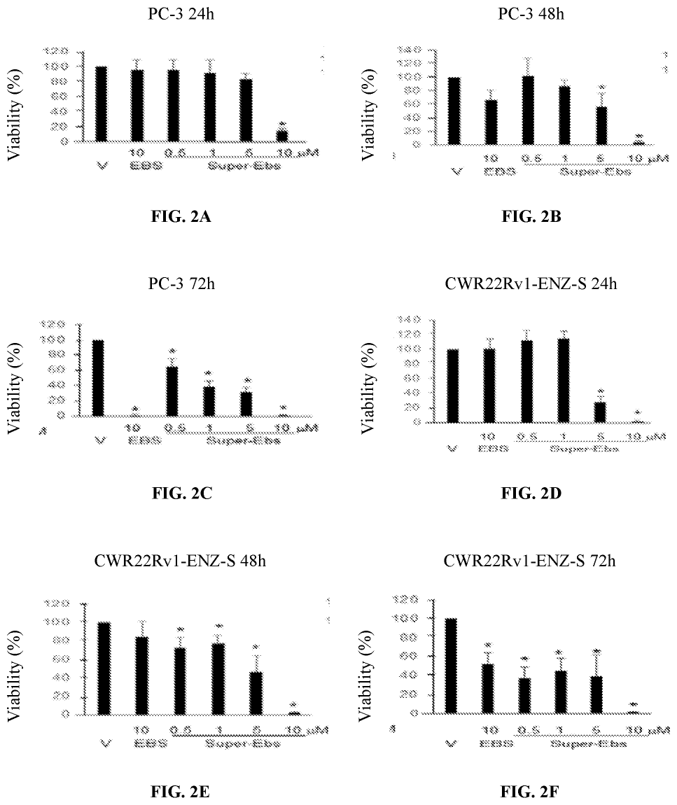

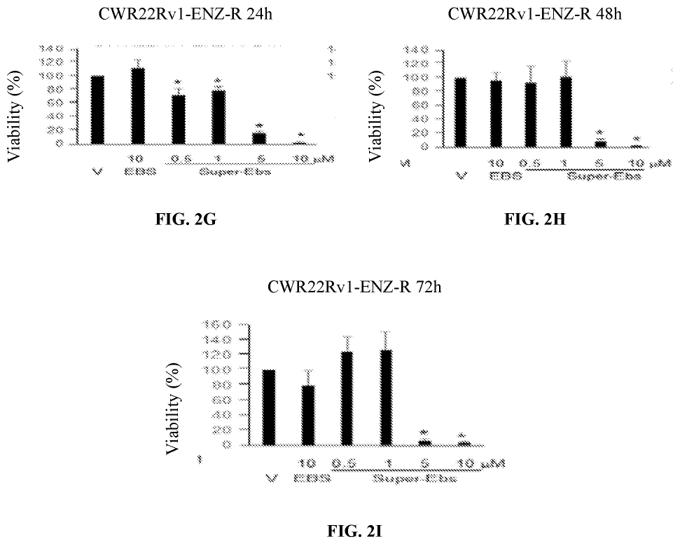

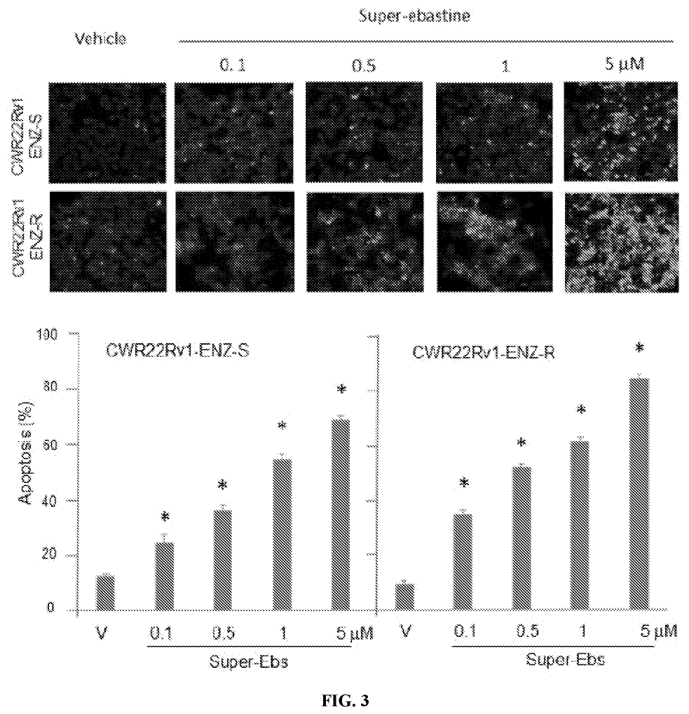

However, high concentrations of EBS (10 μM) were required for growth inhibition of prostate cancer cells. In view thereof, the present inventors chemically modified EBS to identify more potent anti-prostate cancer analogs. This led to the development of a novel analog designated herein as superebastine (Super-EBS) (FIG. 1B). As discussed below, this compound induces apoptosis and growth inhibition of ENZ-resistant, as well as ENZ-sensitive prostate cancer cells at nanomolar concentrations. (FIGS. 2A-3).

Dose and Time Dependent Effect of EBS & Super-EBS on Viability of ENZ-Resistant and -Sensitive Prostate Cancer Cells.

The following prostate cancer cell lines were treated with EBS or Super-EBS: (1) AR negative PC-3; these cells lack AR and are resistant to ABT and ENZ; (2) AR-positive CWR22Rv1 cells, which are ABT-resistant and ENZ-sensitive; and (3) AR-positive CWR22Rv1 cells, which are ABT-resistant and ENZ-resistant. The cells were treated with vehicle, 10 μM EBS, or Super-EBS at 500-, 1-, 5- and 10-μM for 24, 48 and 72 hours. Cell viability was then measured by resazurin assays. As seen in FIGS. 2A-I, PC-3 cells were sensitive to EBS at 10 μM at 72 hours of treatment. On the other hand, Super-EBS (10 μM) was effective in 24 hours. Importantly, Super-EBS was effective at nanomolar concentrations against PC-3 cells (FIGS. 2A-C), CWR22Rv1 ENZ-sensitive (FIGS. 2D-F), and CWR22Rv1 ENZ-resistant cell lines (FIGS. 2G-I). Mean+SD are shown. P<0.01 by Student's t-test.

Super-EBS Causes Cell Death by Activating the Apoptotic Cascade.

After determining that the cells were sensitive to Super-EBS, they were tested to determine whether Super-EBS inhibited cell viability by inducing apoptosis. CWR22Rv1 ENZ-sensitive and CWR22Rv1 ENZ-resistant cell lines were treated with vehicle, 0.1-, 0.5-, 1- and 5-μM of Super-EBS for 24 hours. The cells were then subjected to immunocytochemical (ICC) analysis for active caspase 3 (green fluorescence) to detect apoptotic cells, and 4, 6-diamidino-2-phenylindole (DAPI) staining (blue fluorescence) to reveal the cell nucleus. Cells were scored for apoptosis under a confocal microscope and percent apoptosis was calculated. As seen in FIG. 3, Super-EBS induced significantly higher levels (P<0.01 by Student's t-test) of apoptosis relative to the vehicle control in CWR22Rv1 ENZ-sensitive and CWR22Rv1 ENZ-resistant cell lines. Mean+SD are shown. P<0.01 by Student's t-test.

### Example 2

This Example further describes validation of EBS for treatment of tumor cells. After identifying the anti-histamine Ebastine (EBS) as a small molecule that causes lysosomal catastrophe at high concentrations (LC₅₀ 18-22 μM) in non-small cell lung cancer cells, the effects of EBS on prostate cancer cells were analyzed. High concentrations of EBS (10 μM) caused a maximum of 50% growth inhibition of prostate cancer cell cultures in 24 h. In view thereof, chemical modifications of EBS were performed to identify more potent anti-cancer analogs taking advantage of the fact that advanced PCa cells express an elevated level of a receptor for advanced glycation end products (RAGE). RAGE activation promotes proliferation, invasive bone homing, and metastasis of PCa cells. This EBS analog, designated Super-EBS, induced 95-98% cell death in ENZ-sensitive, as well as ENZ-resistant prostate cancer cell cultures within 24 h. The discussion below describes studies directed towards understanding the molecular mechanism of action of Super-EBS and facilitating the development of a small molecule inhibitor that is cytotoxic against drug-resistant PCa models with the potential for translation for the treatment of drug-resistant CRPC.

Results

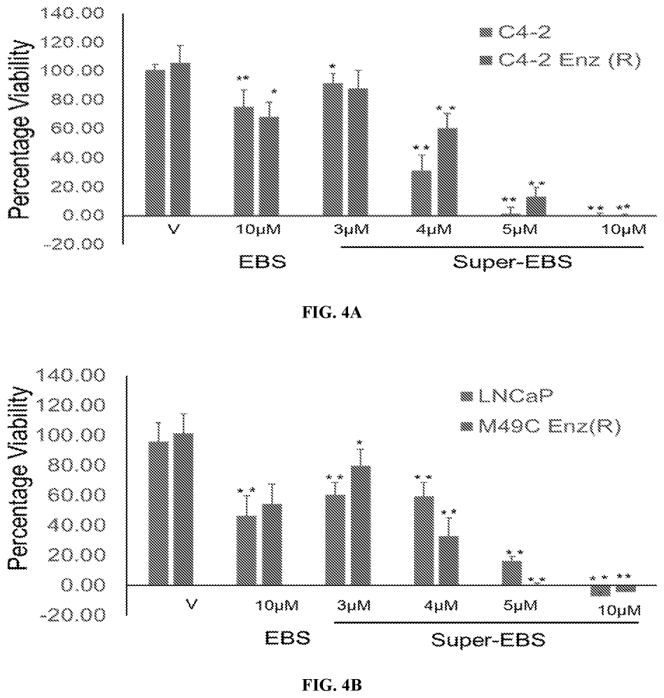

Super-EBS is more effective than EBS in inducing cell death in prostate cancer cells. As seen in FIGS. 4A-B, the cytotoxic effect of Super-EBS (10 μM) was more pronounced than EBS at the same concentration in 24 h. In particular, cell viability of the AR-positive prostate cancer cells and their drug resistant counterparts was lower for the cells treated with Super-EBS. Additionally, the androgen sensitive PCa cell LNCaP and its ENZ-resistant counterpart M49C were more susceptible to the cytotoxic action of Super-EBS than the androgen independent C4-2 and its ENZ resistant counterpart C4-2R.

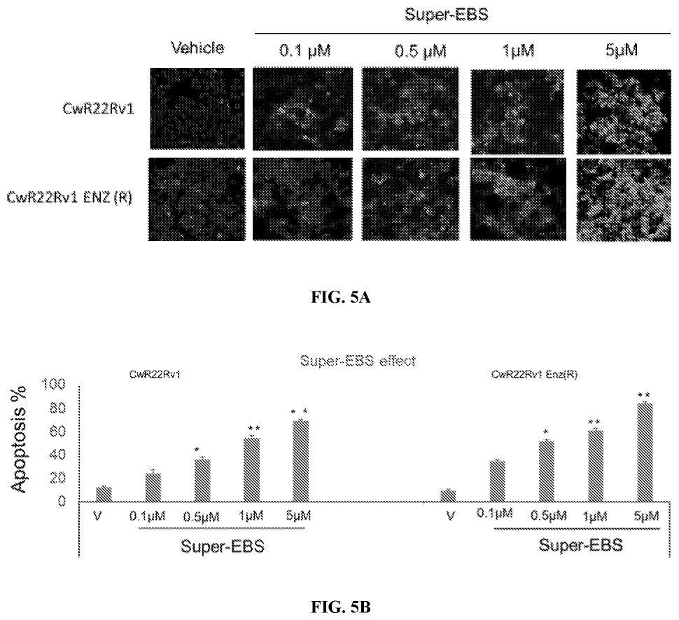

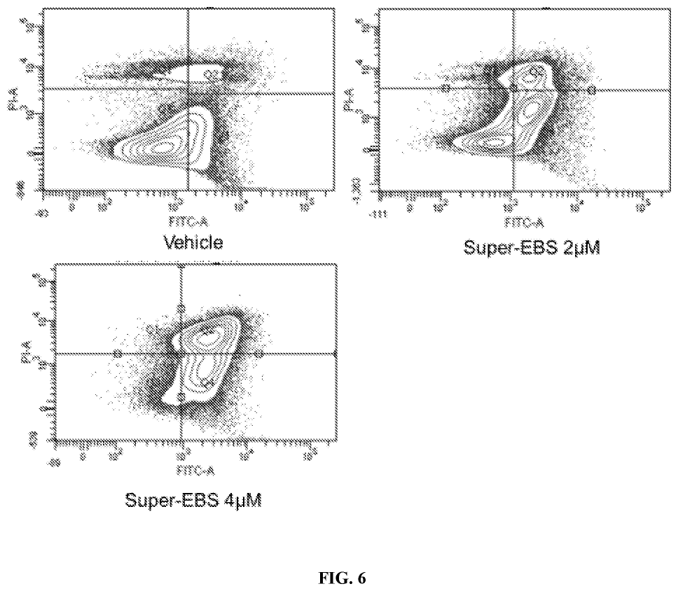

Super-EBS causes cell death by the activation of an apoptotic cascade. Immunocytochemical (ICC) analysis (FIGS. 5A-B) of the AR-v7 expressing CwR22Rv1 and CwR22Rv1 Enz(R) using cleaved caspase 3, a marker for apoptosis, and DAPI, showed a dose dependent increase in apoptotic cells, with highest cell kill noted with 5 μM Super-EBS at 24 h. CwR22Rv1 Enz(R) cells were also tested with Super-EBS for 24 h and subjected to FACS analysis using Annexin V and propidium iodide (PI) staining. A dose dependent increase of cells in the early and late stages of apoptosis was noted post-treatment with Super-EBS (FIG. 6).

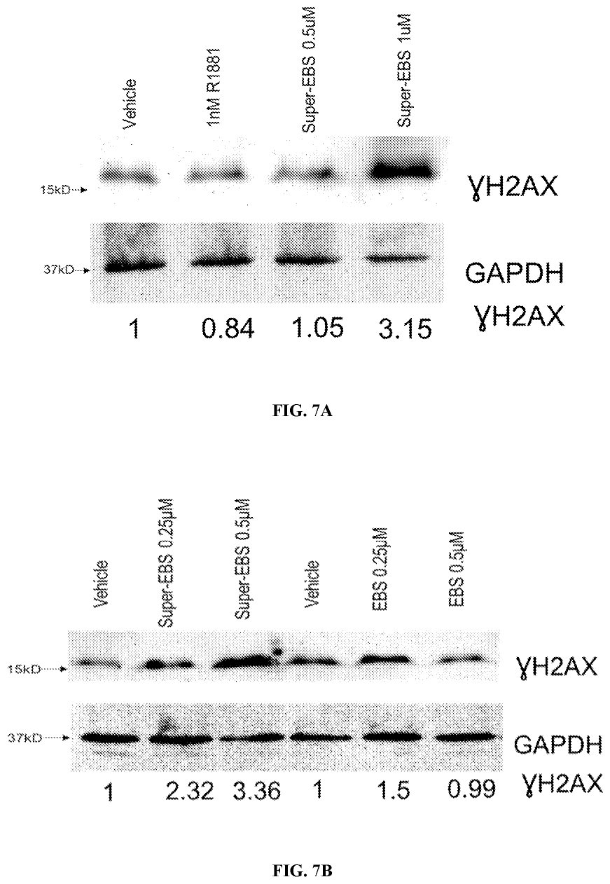

Super-EBS induces DNA damage in AR-positive prostate cancer cells. Lysates from AR-positive androgen-independent C4-2 cells (FIG. 7A) or androgen-dependent LNCaP cells (FIG. 7B) were treated with Super-EBS and subjected to Western blot analysis for γH2AX (Ser 139) positivity to detect DNA double stranded breaks (DSB). Super-EBS induced DSBs in both C4-2 and LNCaP cells (FIGS. 7A-B). It was noted that Super-EBS but not EBS induced significant amount of DSBs (FIG. 7B).

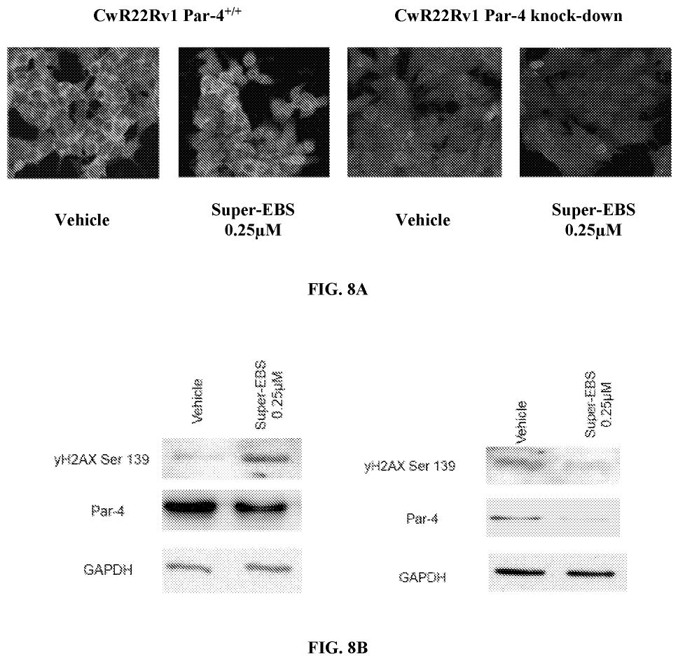

DNA damage induced by Super-EBS is mediated by Par-4. Knock-down of Par-4 in the CwR22Rv1 cells lead to a decrease in the YH2AX positivity, implying inhibition of DSB formation following Super-EBS treatment (FIGS. 8A-B). This suggests that Par-4 is involved in the induction of DNA damage induced by Super-EBS.

### CONCLUSIONS

Based upon the studies above, Super-EBS was found to be more potent than EBS in killing parental and ENZ-resistant prostate cancer cell lines. Additionally, Super-EBS was found to induce DSB formation in both primary and advanced prostate cancer cell lines, whereas EBS at the same concentration does not. This ultimately leads to the induction of apoptosis noted by ICC and FACS analysis. Furthermore, DNA damage in the form of DSB was found to be at least partly dependent on Par-4, as Par-4 knock-down prevented DSB formation.

All publications, patents, and patent applications mentioned in this specification are herein incorporated by reference to the same extent as if each individual publication, patent, or patent application was specifically and individually indicated to be incorporated by reference.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

1. A composition for treating tumor cells, the composition comprising at least one compound having the structure according to Formula 1: 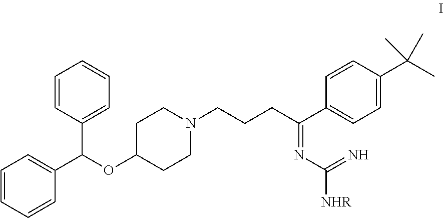
or an analog thereof;
wherein R is selected from the group consisting of H, an alkyl group, a cycloalkyl group, an arylalkyl group, a heteroarylalkyl group, an acyl group, and combinations thereof.

2. The composition of claim 1, wherein the compound comprises the structure:

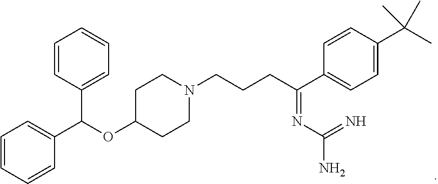 [SMILES: CC(C)(C)c1ccc(/C(CCCN2CCC(OC(c3ccccc3)c3ccccc3)CC2)=N\C(=N)N)cc1]

3. The composition of claim 1, wherein R is an alkyl group.

4. The composition of claim 1, wherein R is a cycloalkyl group.

5. The composition of claim 4, wherein R is a benzylic group.

6. The composition of claim 1, wherein R is an arylalkyl group.

7. The composition of claim 1, wherein R is a heteroarylalkyl group.

8. The composition of claim 1, wherein R is an acyl group.

9. The composition of claim 1, comprising at least two compounds having the structure according to Formula I.

10. The composition according to claim 1, wherein the tumor cells are prostate tumor cells.

11. A method of treating tumor cells, the method comprising administering, to a patient in need thereof, an effective amount of the composition of claim 1.

12. The method of claim 11, wherein the compound comprises the structure:

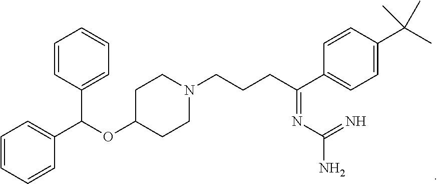 [SMILES: CC(C)(C)c1ccc(/C(CCCN2CCC(OC(c3ccccc3)c3ccccc3)CC2)=N\C(=N)N)cc1]

13. The method of claim 11, wherein R is an alkyl group.

14. The method of claim 11, wherein R is a cycloalkyl group.

15. The method of claim 14, wherein R is a benzylic group.

16. The method of claim 11, wherein R is an arylalkyl group.

17. The method of claim 11, wherein R is a heteroarylalkyl group.

18. The method of claim 11, wherein R is an acyl group.

19. The method of claim 11, wherein the composition comprises at least two compounds having the structure according to Formula I.

20. The method according to claim 1, wherein the tumor cells are prostate tumor cells.
